(19) United States
(12) Patent Application Publication
(10) Pub. No.: US 2014/0180410 A1
(43) Pub. Date: Jun. 26, 2014

(54) INTRAOCULAR LENS OR LENS SYSTEM TO PROVIDE MONOFOCAL VISION AT MULTIPLE PRE-DEFINED FOCAL POINTS

(71) Applicant: NOVARTIS AG, Basel (CH)

(72) Inventor: LAUREN DeVITA GERARDI, CHICAGO, IL (US)

(73) Assignee: NOVARTIS AG, Basel (CH)

(21) Appl. No.: 14/107,425

(22) Filed: Dec. 16, 2013

Related U.S. Application Data
(60) Provisional application No. 61/740,571, filed on Dec. 21, 2012.

(51) Int. Cl. A61F 2/16

(52) U.S. Cl. CPC A61F 2/1635
USPC 623/6.37

(57) ABSTRACT

An accommodative intraocular lens is described that provides monofocal vision at multiple pre-defined focal points during accommodation. The intraocular lens includes a lens element having a series of stacked optics that can be formed utilizing multiple different materials, each of which can have a different stiffness. In an implanted, disaccommodated state, the lens element can have a pre-defined curvature to thus define a first or base focal point of the intraocular lens. Upon accommodation of the patient's eye, the optics can be compressed and/or deformed at differing intervals so as to provide the intraocular lens with three or more distinct focal points. As an alternative, the lens element can include optics formed with multiple sets of haptic elements having varying haptic stiffnesses to vary compression of the optics in order to provide the intraocular lens with multiple focal points.

This application claims the priority of U.S. Provisional Patent Application No. 61/740,571 filed on Dec. 21, 2012.

### FIELD OF THE INVENTION

The invention relates generally to the field of intraocular lens (IOL), and more particularly, to accommodative IOLs for correction of a patient's vision.

### BACKGROUND OF THE INVENTION

Intraocular lenses (IOL) have been developed for implantation in a person's eye to replace the natural crystalline lens which/that has been clouded by cataract, for example. Traditional cataract surgery involving replacement of a patient's natural lens generally has been successful at providing most patients with high quality distance vision. However, in the past, with some types of IOL's, external vision correction (i.e., reading glasses) has been required for near vision tasks, such as reading a computer screen or print in a book.

More recently, IOLs have been developed to provide a more extended depth of focus for cataract patients that can enable adjustment of vision in response to natural accommodation of the patient's eye. For example, a curvature changing IOL can have a particular base refractive power when it is uncompressed (i.e., before implantation into the eye). When implanted in the eye, the IOL ideally should stay in its uncompressed state before the IOL experiences the natural accommodative forces of the eye. However, current IOLs generally can become compressed to some extent when implanted in the eye and remain in such a condition even before the IOLs undergo accommodation, thus undesirably varying or affecting the base refractive power of the IOL. This changes the refractive power of the IOLs which is undesirable because it becomes challenging to determine what the refractive power of the lens is going to be at distance once it is implanted. Additionally, such lenses further can suffer from contrast sensitivity and visual disturbances like halos and glare.

Accordingly, there is a need for an accommodating intraocular lens that provides high quality near, intermediate and distance vision in response to the accommodation of a patient's eye without visual disturbances and without reduced contrast.

### SUMMARY OF THE INVENTION

Briefly described, the present invention generally relates to an intraocular lens that is adapted to be inserted into a patient's eye for adjusting the vision thereof. The intraocular lens can be implanted into the patient's eye, such as within the capsular bag thereof, and generally will include a lens element comprising a first optic, a second optic, a third optic, and a fourth optic generally arranged in stacked series. The optics of the lens element can be formed of various optic materials, such as acrylic, silicone and/or hydrogel materials, having different properties. For example, the second optic can be formed of a material of a first stiffness, while the third optic can be formed of a material of a second stiffness that can be different from the first stiffness of the second optic. In addition, the first and fourth optics can be formed of a material of still a further different stiffness than the first stiffness of the material of the second topic and/or the second stiffness of the material of the third optic. Such varying or different stiffness's of the optics can enable varying degrees or amounts of deformation of the optics to facilitate changes in focal points of the IOL. As implanted, the IOL can have a base refractive power, establishing an initial/first or base focal point of the IOL.

The intraocular lens further generally comprises at least one haptic element connected to the lens element, and projecting radially outward therefrom to a location such that the distal ends thereof will be engaged by portions of the ciliary body of the patient's eye. When the patient's eye undergoes accommodation, the ciliary body generally moves inwardly and/or forwardly, causing the at least one haptic element to be moved axially away from the lens element. Such outward movement of the at least one haptic element, and the further flattening of the capsular bag of the eye during accommodation thereof causes compression of one or more of the optics, with at least the second optic being compressed against the first optic and deformed with respect to the first optic to provide a second, adjusted or intermediate focal point for the IOL. Continued compression of the lens element in response to further accommodation of the patient's eye additionally can cause compression and/or deformation of the third optic with respect to the fourth optic so as to provide further a third, adjusted focal point for the IOL distinct from the first focal point.

As such, an intraocular lens system is described that provides monofocal vision at multiple focal points and may restore pre-presbyopia vision to cataract patients.

Various other objects and advantages of the invention will become apparent to those skilled in the art based on the following drawings and detailed description.

### BRIEF DESCRIPTION OF THE DRAWINGS

FIG. 1 is a schematic view of the intraocular lens 100 in an accommodated state, according to one embodiment of the present invention.

FIGS. 2 and 3 are schematic views of intraocular lens 100 undergoing axial compression, according to one embodiment of the present invention.

FIG. 4 is a schematic view of an intraocular lens 500, according to an alternate embodiment of the present invention.

Those skilled in the art will appreciate and understand that, according to common practice, the various features of the drawings discussed below are not necessarily drawn to scale, and that dimensions of various features and elements of the drawings may be expanded or reduced to more clearly illustrate the embodiments of the present invention described herein.

### DETAILED DESCRIPTION

As illustrated in the drawings, the intraocular lens (IOL) 100, 500, 600 formed according to the principles of the present invention, is designed to provide monofocal vision at multiple focal points during accommodation of a patient's eye. Accommodation is the process by which the eye changes optical power (by changing natural lens curvature or shape) to maintain focus on an object as its distance from the eye changes. This accommodative process is achieved by contraction or relaxation of the ciliary muscles or body of the patient's eye, acting on the capsular bag B so as to cause deformation of the lens of the eye to adjust focus thereof. Specifically, when the ciliary muscles contract (accommodation), the capsular bag is released from tension. When the ciliary muscles relax (disaccommodation), the capsular bag is pulled and flattened by tension in the zonules connecting the ciliary muscles to the capsular bag.

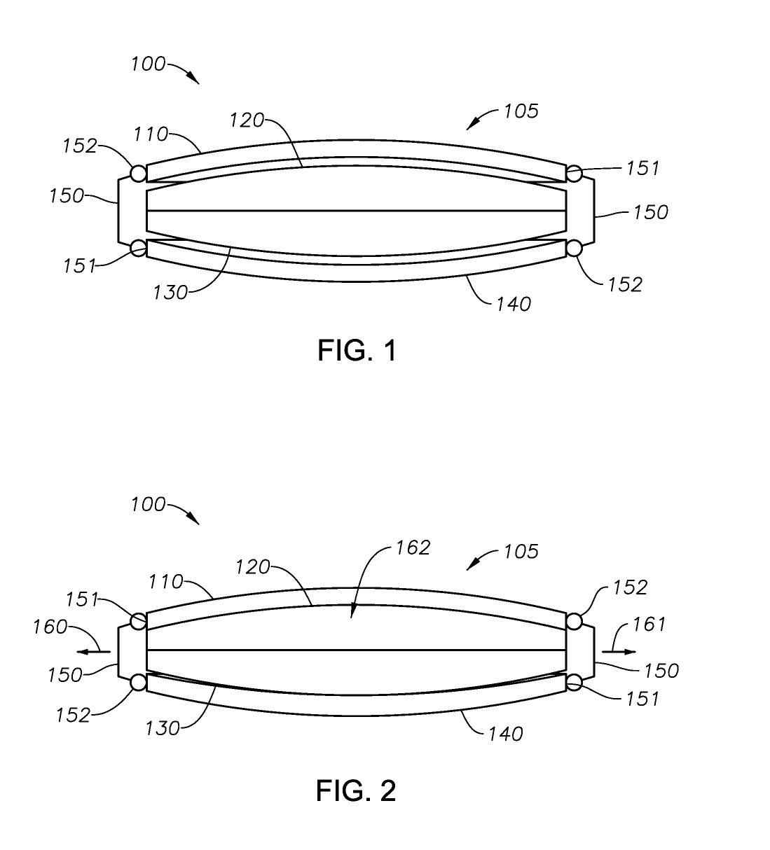

The IOL, illustrated in the Figures in different embodiments 100 (FIGS. 1-3), 500 (FIGS. 4), and 600 (FIG. 5), generally will be implanted within a patient's eye as a replacement for the natural crystalline lens thereof. As illustrated in FIG. 1, in one embodiment, IOL 100 comprises a lens element 105 that includes a first optic 110, a second optic 120, a third optic 130, and a fourth optic 140. The optics 110/120/130/140 generally will be arranged in stacked series, with the first optic 110 and the fourth optic 140 defining an anterior lens component of lens element 105, and the second optic 120 and third optic 130 defining a posterior component of lens element 105 contained within the first and fourth optics. The second optic 120 contacts the first optic 110. The third optic 130 contacts the fourth optic 140. FIG. 1 illustrates IOL 100 in an uncompressed, accommodated or expanded state for providing enhanced focus/visual acuity for distant objects.

The first optic 110, the second optic 120, the third optic 130, and the fourth optic 140 of lens element 105, generally will be formed of soft, flexible, and typically hydrophilic, optical materials, such as silicone, acrylics (for example, AcrySof® acrylic optical material from Alcon Laboratories), hydrogels and/or combinations thereof. The second optic 120 and the third optic 130 further may be formed of or contain a liquid material. In this case, each of the second optic 120 and the third optic 130 further may be encapsulated within a thin, flexible membrane capable of deformation as the fluid material is compressed and/or moved therein.

The second optic 120 generally is formed of a material having a first stiffness and the third optic 130 is formed of a material having a second stiffness. Additionally, the first optic 110 and fourth optic 140 can be formed of a material of a third or different stiffness than the first stiffness of the material of the second optic 120 and the second stiffness of the material of the third optic 130. The first optic 110 and the fourth optic 140 typically will be formed of a material with the highest stiffness among the optics, for example, having a modulus of elasticity on the order of 2+ Megapascals, although greater or lesser stiffness also may be provided and can be of a fixed or negligible base optical power to provide a desired initial correction and/or base or initial focal point when the IOL 100 is in its disaccommodated state. The third optic 130 may be formed of a material with a lower stiffness, such as having a modulus of elasticity in the order of 300-500 Kilopascals, while the second optic 120 may be formed of a material with a least stiffness having a modulus of elasticity in the order of 10-100 Kilopascals, (i.e., stiffness of the material of the third optic can be lower than the stiffness of the material forming the first optic 110 and the fourth optic 140 but higher than the stiffness of the material forming the second optic 120). The optics 110, 120, 130, and 140 may be formed of different materials with different stiffness. Alternatively, the optics 110, 120, 130, and 140 may be formed of the same material having different thicknesses to provide varying stiffness, or the optics 110 and 140 may be formed of a same first material with a desired stiffness, and the optics 120 and 130 may be formed of a same second material with varying thicknesses to provide varying degrees of stiffness and/or resilience.

As further illustrated in FIG. 1, IOL 100 further comprises at least one haptic element 150 connected to a peripheral side edge 106 of lens element 105. Typically, two or more haptic elements 150 will be provided, arranged in substantially equally spaced series about the peripheral side edge 151 of the lens element 105. These haptic elements may be connected to at least one of the first optic 110, the second optic 120, the third optic 130 or the fourth optic 140. In an embodiment, the haptic element(s) may be formed integrally with lens element 105, with the element and lens element molded as a substantially unitary structure. In an alternative embodiment, the haptic element(s) and lens element may be formed as separate pieces or components that are attached together by plasma bonding, adhesives, and/or other bonding techniques. When implanted in the eye, the haptic elements 150 support lens element 105 in an implanted location within or in front of the capsular bag. The haptics 150 act as a force transfer assembly to transfer force to the optics when the capsular bag is either relaxed or stretched by tension in the zonules.

In some embodiments, as illustrated in FIG. 2, a first pair of haptics 150 (location shown in FIG. 1) may be attached to the first optic 110 and the fourth optic 140, such as by an attachment member or bracket, indicated at 152. Additionally, a second pair of haptics also may be attached to the second optic 120 and the third optic 130. The second set of haptics may enable the second optic 120 and the third optic 130 to move relative to the first optic 110 and the fourth optic 140. The haptic elements may be provided with various configurations including plates or arms, Z or S-shaped haptics, or other configurations, and will be engaged by movement of the capsular bag and/or ciliary muscles of the eye (FIG. 1). The haptic elements further will have a shape memory or desired resiliency to apply a compressive force to the optics of the lens element.

Prior to accommodation or when the eye is in a disaccommodated state, IOL 100 generally rests in the capsular bag in a substantially flattened configuration (not otherwise illustrated in the figures) and is held by zonules Z exerting an axial pulling force on the capsular bag when the ciliary muscle or body portions are in a relaxed state. In this state, the at least one haptic element 150 barely contacts the ciliary body, though it may not be affixed to the ciliary body and the ciliary muscles are relaxed, and lens element 105 has its maximum focal length for distant viewing (i.e., a first base or initial focal point for distance vision). Alternatively, the haptics can compress the optics when the capsular bag is flattened and release them when relaxed, in which case the mechanical arrangement can be appropriately modified.

When the eye undergoes accommodation, as indicated in FIGS. 1-3, the ciliary body moves and causes compression of lens element 105, engagement of the ciliary muscles/body with the haptic elements 150. Such engagement in turn causes movement of the haptic elements away from the lens element 105, causing the first optic 110 to move against and compress the second optic 120 thereagainst, resulting in deformation of the second optic 120 with respect to the first optic 110. In other words, the curvature/radius of the second optic 120 is changed and generally conforms to the radius of first optic 110 creating a second, adjusted or intermediate focal point. Further contraction of the ciliary body and further movement of the at least one haptic element causes compression of the third optic 130 against the fourth optic 140 as well (i.e., deformation of the third optic 130 with respect to the fourth optic 140). In other words, radius of the third optic 130 can be substantially conformed to the radius of the fourth optic 140 to create a third, further adjusted or near focal point.

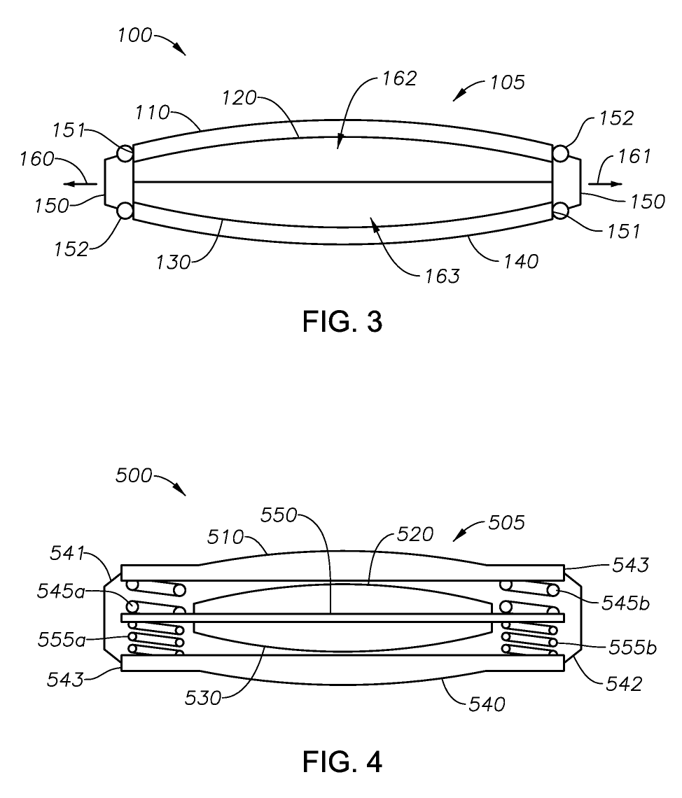

FIGS. 2 and 3 depict IOL 100 with portions of the lens element 105 in a substantially flattened or disaccommodated state, being compressed axially due to the contraction of the ciliary body and outward movement of the haptic elements 150. In particular, FIG. 2 illustrates lens element 105 undergoing a first degree of compression, indicated at arrow 162, which leads to deformation of the second optic 120 with respect to the first optic 110. As the lens element 105 is compressed, the second optic 120, which is formed of material having the lowest degree of stiffness or thickness, is deformed first. As noted, this deformation of the second optic 120 provides the IOL 100 with its second adjusted or intermediate focal point that may provide for improved intermediate vision, such as when looking at a computer screen or somebody's face. FIG. 3 illustrates lens element 105 undergoing a second degree of compression, as indicated at arrow 163, which is generally higher than the first degree of compression and which accordingly leads to deformation of the third optic 130 with respect to the fourth optic. As the lens element 105 is continually compressed (i.e., second degree of compression), the third optic 130 which generally is formed of a stiffer material than the second optic 120, thereafter can be deformed. This deformation of the third optic 130 creates/provides the IOL 100 with its third, further adjusted focal point to adjust the patient's vision to provide additionally enhanced clarity and focus for near or close-up vision, such as for reading a book.

FIG. 4 illustrates an alternative embodiment of an IOL 500 comprising lens element 505. Lens element 505 comprises a first optic 510, second optic 520, third optic 530, fourth optic 540, and a fifth optic 550 arranged in a stacked arrangement. The first optic 510 and fourth optic 540 can define an anterior lens component of lens element 505, while the second optic 520 and third optic 530 can define a posterior component of lens element 505. The fifth optic 550 is located between second optic 520 and third optic 530 and can provide a support between the second and third optics to facilitate their deformation with respect to the first and fourth optics respectively. This fifth optic 550 further can provide a pre-defined optic power for the lens element when in a disaccommodated state.

At least one haptic element 541/542 may be connected to a distal edge 543 of at least one of the first, second, third, or fourth optics. In this embodiment, the at least one haptic element generally will comprise a pair of resilient haptic elements 541/542 attached to the distal edge of at least the first and fourth optics on opposite sides thereof. The haptic elements may comprise resilient elements, here shown as resilient members 545a, 545b, 555a, 555b in the form of curved, circular or “spring-like” elements extending between the first and fourth optics along the distal edges thereof. It will, however, be understood that other configurations of resilient haptic elements having a shape memory and/or desired amount of resilience to apply an expansive force to the optics of lens element 505 as needed also can be used and that the “spring” configuration shown in the drawings is for illustrative principles. As shown in FIG. 5, the resilient haptic elements may comprise a first pair of resilient members 545a, 545b mounted between the first and fifth optic and a second pair of resilient members 555a, 555b mounted between the fourth and fifth optic. The resilient members 545a, 545b separating the first optic 510 and fifth optic 550 are formed of a stiffer material than those separating the fourth optic 540 and fifth optic 550. The resilient members 545a, 545b may be formed of a material of a first stiffness. The resilient members 555a, 555b may be formed of a material of a second stiffness lower than the first stiffness.

The first optic 510, the second optic 520, the third optic 530, the fourth optic 540, and fifth optic 550 of lens element 505 generally will be formed of soft, flexible and typically hydrophilic materials, such as silicone, acrylics (for example, AcrySof®), hydrogels and/or combinations thereof. Also, the resilient haptic elements can be formed from a similar material or a material with greater rigidity, and at least a portion of the haptic elements can be formed with or attached to one or more of the optics of the lens element. The second optic 520 and the third optic 530 may be formed of a liquid material. In this case, each of the second optic 520 and the third optic 530 may be encapsulated within a thin flexible membrane capable of deformation as the fluid material is compressed and/or moved therein.

The first optic 510, fourth optic 540 and fifth optic 550 are formed of a material of a first stiffness. The second optic 520 and the third optic 530 can be formed of a material having a second stiffness or different stiffness than the first stiffness of the material of the first, fourth and fifth optics. The first optic 510, the fourth optic 540, and the fifth optic 550 may be formed of a material with highest stiffness among the optics, for example, having a modulus of elasticity in the order of 2+ Megapascals, although greater or lesser stiffness also may be provided and can be of a fixed or negligible optical power to provide a desired correction and/or focal point when the IOL 500 is in its disaccommodated state. The second optic 520 and third optic 530 may be formed of a material with a lower stiffness (i.e., the stiffness of the material of the second optic 520 and third optic 530 can be different from and generally will be lower than the stiffness of the material forming the first optic 510, the fourth optic 540, and the fifth optic 550).

Prior to accommodation or when the eye is in a disaccommodated state, IOL 500 generally floats in the capsular bag in a substantially flattened configuration, (not otherwise illustrated in the figures) and is held by zonules exerting an axial pulling force on the capsular bag when the ciliary muscle or body portions are in a relaxed state. In this state, the at least one haptic element 541/542 barely contacts the ciliary body, though it may not be affixed to the ciliary body, the ciliary muscles are relaxed, and lens element 505 has its maximum focal length for distant viewing (i.e., a first focal point for distance vision).

When the eye undergoes accommodation, the ciliary body contracts. Contraction of the ciliary body causes compression of lens element 505. Contraction of the ciliary muscles/body causes engagement of the ciliary muscles/body with the at least one haptic element 541/542. Such engagement in turn causes movement of the at least one haptic element away from the lens element 105, causing deformation of at least one of first optic 510, second optic 520, third optic 530, or fourth optic 540. When compressed axially, lens element 505 undergoes a first degree of compression. This causes compression of resilient members 555a, 555b first because they are formed of a material having a reduced stiffness than that of the material forming resilient members 545a, 545b thereby applying compressive forces on the fourth optic 540 and fifth optic 550. The compressive forces cause deformation of third optic 530 with respect to the fourth optic 540 and fifth optic 550. This deformation of the third optic 530 provides an intermediate focus (i.e., second adjusted focal point). When compressed further, the lens element 505 undergoes a second degree of compression. This causes compression of resilient members 545a, 545b as well thereby applying compressive forces on the first optic 510 and fifth optic 550. The compressive forces cause deformation of the second optic 520 with respect to the first optic 510 and fifth optic 550 as well, providing another focus (i.e., third focal point).

As such, the IOL 100/500 provides multiple distinct focal points. Utilizing three or more different materials, each with different stiffness can provide three or more distinct focal points. Alternatively, utilizing two materials but with varying haptic stiffness also provides multiple distinct focal points. When implanted in the eye, IOL 100/500 can have has a pre-defined base power. As long as the sizing of the IOL 100 is right (i.e., not too big to be compressed early when implanted), these distinct focal points can enable desired accommodating changes of the refractive power of the IOL at varying distances unlike existing IOLs.

Simple optical calculations assuming that first optic 110/510, fourth optic 140/540, and fifth optic 550 (for IOLs 100, 500) are of negligible power and assuming an index of refraction for all the materials of 1.5418 suggest the following:

| | Near | Intermediate | Distance |
|---|---|---|---|
| Anterior Deformable | 17 | 20 | 20 |
| Radius (mm) | | | |
| Posterior Deformable | −17 | −17 | −20 |
| Radius (mm) | | | |
| Power of IOL in the eye (D) | 24.2 | 22.4 | 20.6 |

Anterior deformable radius refers to the deformation of the radius/curvature of the second optic. Posterior deformable radius refers to the deformation of the radius/curvature of the third optic. Radii and indices of refraction may be varied to achieve desired near, intermediate and distance refractive powers.

It will be understood that while IOL 100/500 is shown and described in example embodiments as having two to four haptic elements or sets of haptic elements spaced about the periphery of the lens element 105/505, any number of haptic elements may be used to support lens element 105/505 as long as lens element 105/505 is substantially centered with respect to the haptics, without departing from the scope of this disclosure.

The foregoing description generally illustrates and describes various embodiments of the present invention. It will, however, be understood by those skilled in the art that various changes and modifications can be made to the above-discussed construction of the present invention without departing from the spirit and scope of the invention as disclosed herein, and that it is intended that all matter contained in the above description or shown in the accompanying drawings shall be interpreted as being illustrative, and not to be taken in a limiting sense. Furthermore the scope of the present disclosure shall be construed to cover various modifications, combinations, additions, alterations, etc. above and to the above-described embodiments, which shall be considered to be within the scope of the present invention. Accordingly, various features and characteristics of the present invention as discussed herein may be selectively interchanged and applied to other illustrated and non-illustrated embodiments of the invention, and numerous variations, modifications, and additions further can be made thereto without departing from the spirit and scope of the present invention as set forth in the appended claims.

What is claimed is:

1. An intraocular lens comprising:
a lens element comprising a first optic, a second optic, a third optic, and a fourth optic, wherein the second optic is formed of a material of a first stiffness, the third optic is formed of a material of a second stiffness, and the first optic and the fourth optic are formed of a material of a different stiffness than the first stiffness of the material of the second optic and the second stiffness of the material of the third optic;
at least one haptic element connected to the lens element, wherein movement of the at least one haptic element causes compression of at least the second optic against the third optic; and
wherein deformation of the second optic with respect to the first optic provides a first focal point and deformation of the third optic with respect to the fourth optic provides a second focal point distinct from the first focal point.

2. The intraocular lens of claim 1, wherein the second optic contacts the first optic and the third optic contacts the fourth optic.

3. The intraocular lens of claim 1, wherein the second optic undergoes deformation due to a first degree of compression of the lens element, and wherein the third optic undergoes deformation due to a second degree of compression of the lens element.

4. The intraocular lens of claim 3, wherein the second degree of compression is higher than the first degree of compression.

5. The intraocular lens of claim 3, wherein compression of the lens element is caused by contraction of a ciliary body of the wearer's eye in which the intraocular lens is placed.

6. An intraocular lens comprising:
a lens element comprising a first optic, a second optic, a third optic, and a fourth optic arranged in a stacked arrangement, the first and fourth optics defining an anterior lens component and the second and third optics defining a posterior component;
at least one haptic element connected to a distal edge of at least one of the first, second, third or fourth optics wherein movement of the at least one haptic element causes deformation of at least one of the first, second, third or fourth optics;
wherein the first optic and the fourth optic are formed of a material of a first stiffness, and the second optic and the third optic are formed of a material of a second stiffness; and
wherein deformation of the second optic with respect to the first optic provides a first focal point and deformation of the third optic with respect to the fourth optic provides a second focal point distinct from the first focal point.

7. The intraocular lens of claim 6, further comprising a fifth optic located between the second and third optics.

8. The intraocular lens of claim 7, wherein the fifth optic is formed from the material having the first stiffness.

9. The intraocular lens of claim 6, wherein the at least one haptic element comprises a pair of haptic elements attached to the distal side edge of at least the first and fourth optics on opposite sides thereof.

10. The intraocular lens of claim 9, wherein the haptic elements comprise spring members extending between the first and fourth optics along the distal side edges thereof.

12. The intraocular lens of claim 9, wherein the haptic elements comprise a first pair of spring members mounted between the first and fifth optic and a second pair of spring members mounted between the fourth and fifth optic.

13. The intraocular lens of claim 12, wherein the first pair of spring members is formed of a material with a first stiffness and the second pair of members is formed of a material with a second stiffness lower than the first stiffness.
